(19) United States
(12) Patent Application Publication
(10) Pub. No.: US 2023/0190148 A1
(43) Pub. Date: Jun. 22, 2023

(54) METHOD AND APPARATUS FOR TEMPERATURE COMPENSATION OF LOW BATTERY VOLTAGE THRESHOLDS AND VOLTAGE DROOP DETECTION IN A MEDICAL DEVICE

(71) Applicant: Roche Diabetes Care, Inc., Indianapolis, IN (US)

(72) Inventors: Michael Joseph Blackburn, Indianapolis, IN (US); Duane Fridley, Indianapolis, IN (US); Kurt Klem, Indianapolis, IN (US); Larry L. Lockridge, Fishers, IN (US); Justin Brett Lybbert, Fishers, IN (US); Nathan Eugene Manlove, Noblesville, IN (US); Joe Brown Thomas, Greenfield, IN (US)

(73) Assignee: Roche Diabetes Care, Inc., Indianapolis, IN (US)

(21) Appl. No.: 18/169,347

(22) Filed: Feb. 15, 2023

Related U.S. Application Data
(63) Continuation of application No. PCT/US2021/046844, filed on Aug. 20, 2021.
(60) Provisional application No. 63/068,633, filed on Aug. 21, 2020.

(51) Int. Cl. A61B 5/145

(52) U.S. Cl. CPC A61B 5/14532; A61B 2560/0204

(57) ABSTRACT

A method for operating a medical device includes activating a processor that receives electrical power from a battery in the medical device, measuring a temperature within a housing of the medical device, identifying a low battery voltage threshold based on the temperature, measuring a first voltage level of the battery, commencing an operation sequence after measuring the first voltage level of the battery, generating a plurality of voltage comparisons between a reference voltage level and a voltage level delivered from the battery during the operation sequence, and generating, an output indicating a low battery condition if at least one of the first voltage level of the battery is less than the low battery voltage threshold and above a predetermined minimum operating voltage threshold, or at least one voltage comparison indicating the voltage level of the battery is less than the reference voltage level during the operation sequence.

### CLAIM OF PRIORITY

This application is a continuation of PCT/US2021/046844, filed Aug. 20, 2021, which claims priority to U.S. Provisional Application No. 63/068,633, filed on Aug. 21, 2020, the entire contents of which are hereby incorporated herein by reference.

### TECHNICAL FIELD

The disclosure relates generally to the field of battery powered medical devices and, more specifically, to battery powered medical devices, including blood glucose test meters.

### BACKGROUND

Analyte test meters that are known to the art enable the analysis of a bodily fluid sample provided by a user to identify the level of one or more analytes in the body of the user using an electronic device and one or more electrochemical reactions. These analyte meters provide significant benefits for the accurate measurement of analytes in fluidic samples (i.e., biological or environmental) for individual users. An analyte meter applies electrical signals to the combination of the reagents and the fluid sample and records responses to the applied electrical signals. A combination of electronic hardware and software in the analyte test meter implements a detection engine that detects a level of the analyte in the body of the user based on the recorded responses to the electrical signals. For example, persons with diabetes can benefit from measuring glucose by providing a fluid sample of blood or another bodily fluid to reagents that are formed on an electrochemical test strip, which is electrically connected to a blood glucose meter (BGM). The BGM provides a measurement of the blood glucose level of the user, and many BGM devices use single-use electrochemical test strips that are discarded after each blood glucose measurement. Analyte test meters can also provide benefits to users at-risk for heart disease by providing measurements of cholesterols and triglycerides, among other analytes. These are but a few examples of the benefits of measuring analytes in biological samples. Advancements in the medical sciences are identifying a growing number of analytes that can be electrochemically analyzed in a fluidic sample.

Many existing analyte test meters use batteries as an energy source to power the electronic components of the analyte meters and to provide a compact and portable test meter that a person with diabetes (PwD) or other medical user carries about his or her person. In typical use, the test meter is activated for use over a comparatively short period, often one minute or less for obtaining a blood glucose measurement, during which one or more batteries provide electrical current to operate components in the test meter. The test meter undergoes comparatively prolonged periods in a deactivated or “hibernation” mode in which the test meter is inactive and the batteries in the test meter provide little or no electrical current to the test meter. For example, even in a heavy usage scenario in which a PwD performs ten tests of his or her blood sugar during a day, the blood glucose meter spends the large majority of the day in the deactivated mode, and many blood glucose meters experience lower-frequency usage that produces even longer periods of inactivity. For example, some PwDs test blood sugar only three times per day, and some PwDs who employ continuous glucose monitors (CGMs) only use a portable blood glucose meter on an infrequent basis (e.g. once every few days or even weeks/months) to verify and supplement data from a CGM.

During idle periods, the internal temperature of the BGM may change as the BGM is transported to different environments that may subject the BGM to colder or hotter temperatures for prolonged periods of time. The changes in temperature may affect the nominal voltage of one or more batteries in the BGM, especially when the BGM activates from a prolonged hibernation state in which the batteries were not being monitored to detect if any discharge has occurred. Variations in temperature may lead to a false detection of a low battery condition or a failure to detect a low battery condition depending upon the temperature. Furthermore, during operation of the BGM the nominal battery voltage that is measured in an idle or lightly-loaded state may not provide enough information to identify all low battery conditions that may occur during operation of the BGM when the battery operates under a higher load condition. Consequently, improvements to blood glucose meters and other battery powered medical devices that detect low battery conditions over a wide range of operating temperatures and during operating sequences would be beneficial.

### SUMMARY

In one embodiment, a method for operating a medical device includes activating a processor in the medical device, the processor receiving electrical power from a battery electrically connected to the medical device, measuring, with the processor, a temperature within a housing of the medical device, identifying, with the processor, a first low battery voltage threshold based on the temperature, measuring, with a voltage sensor operatively connected to the processor, a first voltage level of the battery, commencing an operation sequence of the medical device after measuring the first voltage level of the battery, generating, with a voltage comparator operatively connected to the processor, a plurality of voltage comparisons between a reference voltage level and a voltage level delivered from the battery during the operation sequence, and generating, with the processor, an output using an output device in the medical device indicating a low battery condition in response to at least one of the first voltage level of the battery being less than the first low battery voltage threshold and above a predetermined minimum operating voltage threshold, the predetermined operating voltage threshold being less than the first low battery voltage threshold, or at least one voltage comparison in the plurality of voltage comparisons indicating the voltage level of the battery is less than the reference voltage level during the operation sequence.

In another embodiment, a method for operating a medical device includes activating a processor in the medical device, the processor receiving electrical power from a primary battery electrically connected to the medical device, activating, with the processor, at least one peripheral device in the medical device, the at least one peripheral device receiving electrical power from a secondary battery electrically connected to the medical device, measuring, with the processor, a temperature within a housing of the medical device, identifying, with the processor, a first low battery voltage threshold based on the temperature, identifying, with the processor, a second low battery voltage threshold based on the temperature, measuring, with a voltage sensor operatively connected to the processor, a first voltage level of the primary battery, measuring, with the voltage sensor operatively connected to the processor, a second voltage level of the secondary battery, and generating, with the processor, an output using an output device in the medical device indicating a low battery condition in response to at least one of the first voltage level of the primary battery being less than the first low battery voltage threshold and above a first predetermined minimum operating voltage threshold of the primary battery, the first predetermined operating voltage threshold being less than the first low battery voltage threshold, or the second voltage level of the secondary battery being less than the second low battery voltage threshold and above a second predetermined minimum operating voltage threshold of the secondary battery, the second predetermined operating voltage threshold being less than the second low battery voltage threshold.

In another embodiment, a method for operating a medical device includes activating a processor in the medical device, the processor receiving electrical power from a battery electrically connected to the medical device, commencing an operation sequence of the medical device, generating, with a voltage comparator operatively connected to the processor, a plurality of voltage comparisons between a reference voltage level and a voltage level delivered from the battery during the operation sequence, and generating, with the processor, an output using an output device in the medical device indicating a low battery condition in response to at least one voltage comparison in the plurality of voltage comparisons indicating the voltage level of the battery is less than the reference voltage level during the operation sequence.

In another embodiment, a method for operating a medical device includes activating a processor in the medical device, the processor receiving electrical power from a battery electrically connected to the medical device, measuring, with the processor, a temperature within a housing of the medical device, identifying, with the processor, a first low battery voltage threshold based on the temperature, measuring, with a voltage sensor operatively connected to the processor, a first voltage level of the battery, and generating, with the processor, an output using an output device in the medical device indicating a low battery condition in response to the first voltage level of the battery being less than the first low battery voltage threshold and above a first predetermined minimum operating voltage threshold of the battery, the first predetermined operating voltage threshold being less than the first low battery voltage threshold.

### BRIEF DESCRIPTION OF THE DRAWINGS

The advantages, effects, features and objects other than those set forth above will become more readily apparent when consideration is given to the detailed description below. Such detailed description makes reference to the following drawings, wherein:

FIG. 1 is a schematic diagram of a battery-powered medical device, which is further depicted as a blood glucose monitor that operates using a single battery.

FIG. 2 is a schematic diagram of a battery-powered medical device, which is further depicted as a blood glucose monitor that operates using two batteries.

FIG. 3 is a graph depicting a temperature-dependent low battery threshold function for a primary battery.

FIG. 4 is a graph depicting another temperature-dependent low battery threshold function for a secondary battery.

FIG. 5 is a graph depicting an example of voltage droops that are detected during a series of operations of a battery-powered medical device.

FIG. 6 is a block diagram of a process for detection of low battery conditions during operation of the battery-powered medical devices of FIG. 1 and FIG. 2.

### DETAILED DESCRIPTION

These and other advantages, effects, features and objects are better understood from the following description. In the description, reference is made to the accompanying drawings, which form a part hereof and in which there is shown by way of illustration, not limitation, embodiments of the inventive concept. Corresponding reference numbers indicate corresponding parts throughout the several views of the drawings.

While the inventive concept is susceptible to various modifications and alternative forms, exemplary embodiments thereof are shown by way of example in the drawings and are herein described in detail. It should be understood, however, that the description of exemplary embodiments that follows is not intended to limit the inventive concept to the particular forms disclosed, but on the contrary, the intention is to cover all advantages, effects, and features falling within the spirit and scope thereof as defined by the embodiments described herein and the embodiments below. Reference should therefore be made to the embodiments described herein and embodiments below for interpreting the scope of the inventive concept. As such, it should be noted that the embodiments described herein may have advantages, effects, and features useful in solving other problems.

The devices, systems and methods now will be described more fully hereinafter with reference to the accompanying drawings, in which some, but not all embodiments of the inventive concept are shown. Indeed, the devices, systems and methods may be embodied in many different forms and should not be construed as limited to the embodiments set forth herein; rather, these embodiments are provided so that this disclosure will satisfy applicable legal requirements.

Likewise, many modifications and other embodiments of the devices, systems and methods described herein will come to mind to one of skill in the art to which the disclosure pertains having the benefit of the teachings presented in the foregoing descriptions and the associated drawings. Therefore, it is to be understood that the devices, systems and methods are not to be limited to the specific embodiments disclosed and that modifications and other embodiments are intended to be included within the scope of the embodiments. Although specific terms are employed herein, they are used in a generic and descriptive sense only and not for purposes of limitation.

Unless defined otherwise, all technical and scientific terms used herein have the same meaning as commonly understood by one of skill in the art to which the disclosure pertains. Although any methods and materials similar to or equivalent to those described herein can be used in the practice or testing of the methods, the preferred methods and materials are described herein.

Moreover, reference to an element by the indefinite article “a” or “an” does not exclude the possibility that more than one element is present, unless the context clearly requires that there be one and only one element. The indefinite article “a” or “an” thus usually means “at least one.” Likewise, the terms “have,” “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. For example, the expressions “A has B,” “A comprises B” and “A includes B” may refer both to a situation in which, besides B, no other element is present in A (i.e., a situation in which A solely and exclusively consists of B) or to a situation in which, besides B, one or more further elements are present in A, such as element C, elements C and D, or even further elements.

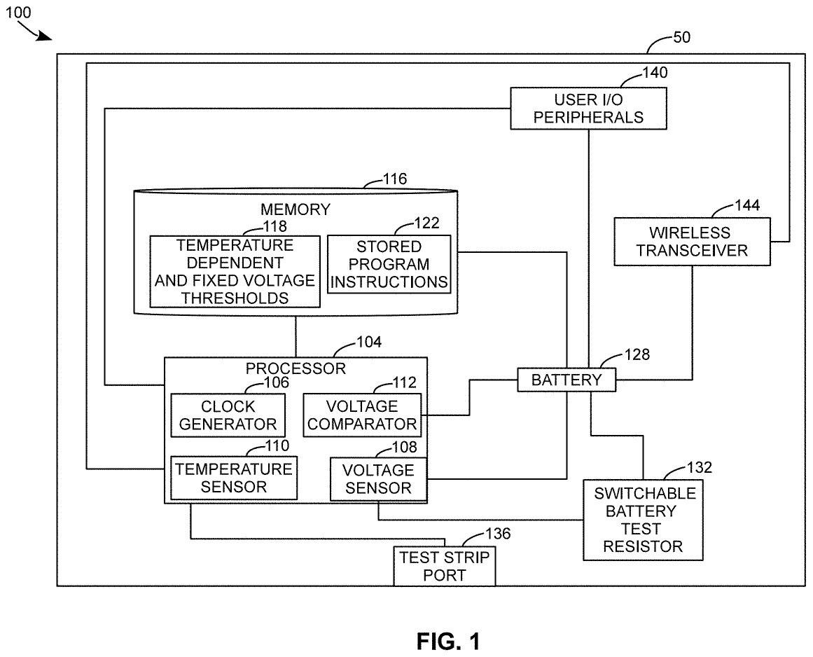

FIG. 1 depicts a schematic diagram of a battery-powered medical device 100 that is configured to identify low battery conditions over a range of operating temperatures and during an operation sequence. A housing 50 in the medical device 100 includes a receptacle for a replaceable battery 128 that is electrically connected to the medical device 100 and houses the other components of the medical device 100. The medical device 100 operates using electrical power delivered from the battery 128 to operate a processor 104 memory 116, user input/output (I/O) peripherals 140, and a wireless transceiver 144 peripheral device. In the illustrative embodiment of FIG. 1, the battery 128 is a single lithium battery that is available commercially as a CR2032 coin cell battery with a nominal 3 V voltage level in a fully-charged battery. However, in alternative embodiments the battery 128 is a different type of battery. Furthermore, in alternative embodiments what is referred to as the single battery 128 further includes multiple battery cells that are electrically connected in a series, a parallel, or a series-parallel configuration to act as a power source for components in a medical device. In the illustrative example of FIG. 1, the medical device 100 is a blood glucose meter that includes a test strip port 136. The test strip port 136 receives a portion of an electrochemical test strip and provides electrical connections between electrodes in the test strip and the processor 104 to enable the processor 104 to apply signals in an electrical test sequence and receive response signals from the test strip to enable measurement of glucose levels in a blood sample that is applied to the test strip 136. Other medical device embodiments that do not perform blood glucose measurements or other forms of electrochemical analyte measurement do not include the test strip port 136.

In the medical device 100, the processor 104 includes one or more digital logic devices such as a microcontroller, microprocessor, application specific integrated circuit (ASIC), or any other electronic device or devices that implement the digital logic functions to perform the operations for detecting low battery conditions and for operation of the medical device 100. While not depicted in further detail herein, the processor 104 also incorporates or is operatively connected to digital-to-analog converters, drive signal generators, signal measurement circuits, and analog-to-digital converters and any other electronic components that are required for the processor 104 to generate an electrical test sequence that is applied to electrodes in an electrochemical test strip through the test strip port 136 and for the processor 104 to detect electrical response signals from the electrochemical test strip in response to the electrical test sequence. While not depicted in greater detail, the processor 104 also includes input/output (I/O) hardware that operatively connects the processor 104 to the I/O peripherals 140, the wireless transceiver 144, and the memory 116.

In the medical device 100, the processor 104 is operatively connected to a clock generator 106, voltage sensor 108, temperature sensor 110, and a voltage comparator 112. In the illustrative embodiment of FIG. 1, the processor 104 incorporates the clock generator 106, voltage sensor 108, temperature sensor 110, and the voltage comparator 112 in a system-on-a-chip configuration to implement the operative connection, although in other configurations these components are separate and the processor 104 is operatively connected to them via a peripheral interconnection interface such as I²C, SPI, RS-232/RS-485, PCI or PCIe, or any other suitable peripheral interconnection.

In the medical device 100, the clock generator 106 includes an oscillator and other electronic components that are generally known to the art to generate a clock signal that synchronizes the execution of operations of the processor 104. The clock generator 106 generates clock signals with at least two different frequencies that adjusts the speed of execution of instructions in the processor 104, which in turn affects the level of electrical power consumption of the processor 104 during operation with lower-frequency clock speeds drawing lower power levels than higher-frequency clock speeds. In one configuration, the processor 104 operates in a low-power operating mode with the clock generator 106 producing a 1 MHz clock signal and the processor 104 operates in an increased-power operating mode with the clock generator 106 producing a 16 MHz clock signal. Of course, alternative processor configurations employ clock generators that produce different specific clock frequencies and clock generators are configurable to generate clock signals at three or more different frequencies as well.

In the medical device 100, the voltage sensor 108 includes an analog voltage measurement device and an analog-to-digital converter (ADC) that provides digital data corresponding to the voltage of the battery 128 to the processor 104. The voltage sensor 108 is operatively connected to the battery 128 and to a switchable battery test resistor 132. The voltage sensor 108 detects a voltage across the terminals of the battery 128 both when the battery 128 is minimally loaded and when the battery 128 is connected to the switchable battery test resistor 132. The switchable battery test resistor 132 includes a resistor with a predetermined resistance level (e.g. 820Ω) that applies a high-impedance load across the terminals of the battery 128. The high-impedance load draws minimal current from the battery 128, but enables the voltage sensor 108 to measure both an open-circuit and loaded voltage level of the battery 128. The processor 104 operates a switch, such as a solid-state switching transistor or relay, to connect the resistor to the battery 128 to enable the voltage sensor 108 to measure the voltage of the battery 128 while under a predetermined load and to disconnect the battery test resistor 132 from the battery 128 after measuring the voltage. In the embodiments of the medical device 100 and the medical device 200 described herein, the ADC in the voltage sensor 108 is connected to different components within the medical device using a multiplexer or other suitable switching device during different portions of operation sequences in the medical device. For example, the ADC converts analog voltage levels of electrical signal responses from electrodes in a test strip that is inserted in the test strip port 136 to digital data for the processor 104 during different portions of an analyte measurement operation sequence. As such, the voltage sensor 108 is not available for use in performing battery voltage measurements during times when the ADC is connected to different components within the medical device.

In the medical device 100, the temperature sensor 110 is a thermocouple, thermistor, resistance thermometer (RTD), solid-state temperature sensor, or any other suitable device that enables the processor 104 to measure temperature levels electronically. Suitable temperature sensing devices are generally known to the art and are not described in further detail herein. In the configuration of the medical device 100, the temperature sensor 110 provides an internal temperature measurement corresponding to components, including the battery 128, that are inside of the housing 50 of the medical device 100 and this temperature measurement is not necessarily equivalent to the ambient air temperature in the environment surrounding the medical device 100. In general, the interior of the medical device 100 is compact and the components within the medical device 100 assume generally uniform temperatures when the medical device 100 is deactivated or in a low-power operating state. As such, the processor 104 is configured to receive a temperature measurement from the temperature sensor 110 within the housing 50 of the medical device 100 and use the temperature measurement to identify the internal temperature of the battery 128, which the processor 104 further uses to identify the low battery voltage threshold for the battery 128 as described in further detail below.

In the medical device 100, the voltage comparator 112 is a sensor that compares a predetermined reference voltage to a supply voltage that is received from the battery 128. The voltage comparator is, for example, an operational amplifier (Op-Amp) or other suitable circuit that has a first input for a reference voltage signal and a second input that receives the voltage from the battery. The reference voltage is generated by, for example, by a digital-to-analog converter (DAC) utilizing a resistor ladder network, which produces an analog voltage level that is generally below the voltage level delivered from the battery 128 during operation, although the precise voltage level of the reference voltage is not necessary set to a fixed threshold. In the medical device 100, if the voltage delivered from the battery 128 drops below the reference voltage level during operation of the medical device 100, then the voltage comparator 112 generates an output that indicates a voltage droop has occurred, although the voltage comparator does not determine the magnitude of how much the voltage droop is less than the reference voltage level. In the embodiment of FIG. 1, the voltage comparator 112 is gated by the clock signals from the clock generator 106, and the voltage comparator 112 identifies if a voltage droop in the battery 128 occurs during a single clock cycle, where zero, one, or multiple voltage droops may occur over a series of clock cycles. The output of the voltage comparator 112 sets a binary status flag or a counter to enable the processor 104 to identify the detection of one or more voltage droops over a series of clock cycles. The voltage comparator 112 detects transient voltage droops in the voltage level of the battery 128 while the battery 128 receives varying loads during operation of the medical device 100 device more quickly and efficiently than the voltage sensor 108. However, the voltage comparator 112 does not generate precise voltage measurements and only detects if a transient voltage droop has occurred during a clock cycle. By contrast, the voltage sensor 108 generates precise voltage level measurements of the battery 128 during device startup and other low-load states in which the battery 128 is at or near a quiescent state, but as described above the ADC in the voltage sensor 108 is connected to different components in the medical device during different portions of an operation sequence, while the voltage comparator 112 remains connected to the battery 128 during the operation sequence.

In the medical device 100, the memory 116 is a digital data storage device that includes at least one non-volatile data storage device such as an EEPROM, NAND or NOR flash, phase change memory, or other suitable data storage devices that retain stored digital data in the absence of electrical power from the battery 128. The memory 116 further includes one or more volatile memory devices including a static or dynamic random-access memory (RAM) that either is integrated into the processor 104 or is embodied as a separate memory device. The memory 116 holds a set of battery voltage thresholds 118, and stored program instructions 122 that the processor 104 executes to perform the low battery detection operations and other functions of a medical device that are described herein.

The battery voltage thresholds 118 include both fixed and temperature-dependent low battery voltage threshold data that the processor 104 uses to determine the state of the battery 128 based on voltage measurements received from the voltage sensor 108. In the embodiment of FIG. 1, the fixed battery voltage thresholds include a minimum operating voltage threshold that is required for the medical device 100 to perform normal operations and a fixed dead battery voltage threshold beneath which the battery 128 is considered to be discharged to the point that the medical device 100 shuts down without any further operation. In one non-limiting configuration, the minimum operating voltage threshold is approximately 2.46V while the dead battery voltage is approximately 2.40V. If the battery 128 exhibits a nominal voltage that is less than the minimum operating voltage threshold but greater than the dead battery voltage, then the processor 104 generates an error indicating the need to replace the battery 128 using a display screen, indicator light, or other output device in the user I/O peripherals 140, and the medical device 100 does not continue with any other operations such as generating blood glucose measurements. If the voltage of the battery 128 is less than the dead voltage threshold then the processor 104 immediately shuts down the medical device 100 without producing a battery replacement output.

The battery voltage thresholds 118 also include temperature-dependent low battery voltage thresholds that are higher than the predetermined minimum battery operating voltage and that the processor 104 uses to identify a low battery condition. If the voltage of the battery 128 is above the temperature-dependent low battery threshold, then the medical device 100 continues with a standard operation sequence. If, however, the processor 104 identifies that the voltage of the battery 128 is below the temperature-dependent low battery threshold, then the processor 104 generates an output to indicate a low battery condition using a display screen, indicator light, or other output device in the user I/O peripherals 140, although the medical device 100 continues with normal operation because the voltage of the battery 128 still exceeds the minimum operating voltage threshold.

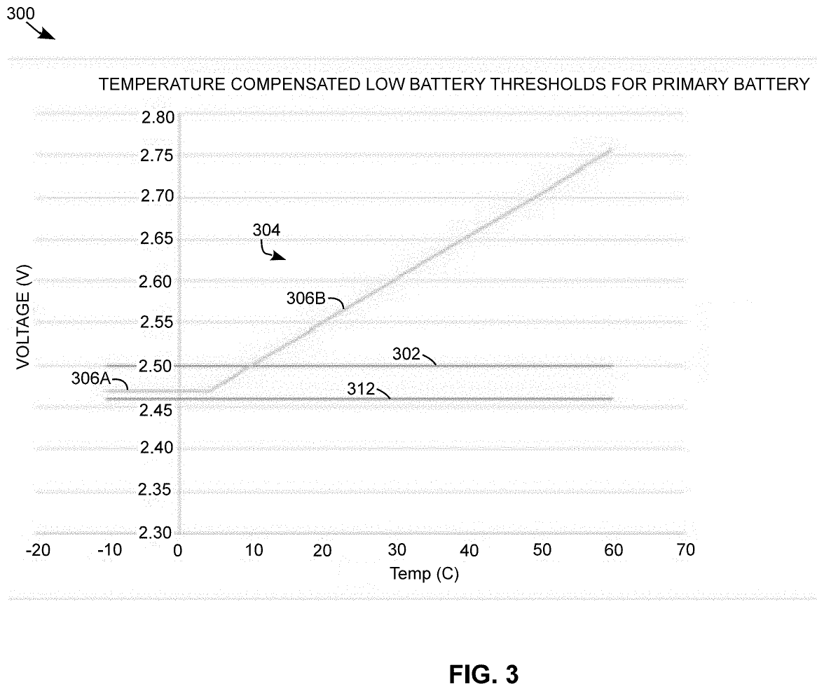

In one configuration, the medical device 100 uses a piecewise linear function to implement the temperature-dependent low battery voltage thresholds. FIG. 3 depicts a graph 300 of an example of a piecewise linear function for a primary battery in a medical device, such as the battery 128 in FIG. 1. In the graph 300, the temperature-dependent low battery threshold 304 is a piecewise linear function that includes a first segment 306A that establishes a low battery threshold voltage of approximately 2.46V for colder operating temperatures in a range of −10 C to 5 C. A second segment 306B is another linear segment with a positive slope in relation to the operating temperature that increases the low battery voltage threshold level as the temperature increases from 5 C to 60 C in the operating temperature ranges of the medical device 100. FIG. 3 depicts an operating temperature range of −10 C to 60 C for the medical device 100. As noted above, these temperatures correspond to internal temperatures measured within the medical device 100 and are not necessarily identical to the ambient air temperature around the medical device 100 during operation. As such, a 60 C temperature can, for example, correspond to an internal temperature in the medical device 100 when it has been stored in a vehicle during summertime even if the ambient air temperature is not 60 C.

FIG. 3 also depicts a prior-art fixed low battery voltage threshold 302 for illustrative purposes, although the medical device 100 does not use the fixed low battery voltage threshold 302. During operation, if the processor 104 and voltage sensor 108 measure a battery voltage level that exceeds the temperature-dependent low battery threshold 304, then the medical device 100 continues with normal operation, while any voltage measurement that is below the temperature-dependent low battery voltage threshold 304 at the measured temperature but also above the minimum operating voltage threshold 312 enables the medical device 100 to continue with normal operation while the processor 104 generates a low battery indicator to alert the user that the battery 128 is approaching the point of replacement. The temperature-dependent low battery threshold 304 is lower than the fixed low battery threshold 302 at lower temperatures of −10 C up to 10 C, and is higher than the fixed low battery threshold 302 at higher temperatures above 10 C up to 60 C. As such, the temperature-dependent low voltage threshold 304 reduces the occurrences of false-positive low battery voltage detections at lower temperatures and reduces the occurrences of false-negative failures to detect low battery conditions at higher temperatures. For every temperature range, the temperature-dependent low battery threshold 304 is higher than the minimum operating voltage threshold 312.

In the medical device 100, the memory 116 stores parameters that describe the piecewise linear functions, such as slope, Y-intercept, and breakpoints between segments of the piecewise linear functions, and the processor 104 calculates the low battery voltage threshold using a temperature measurement from the temperature sensor 110 as the independent variable in the piecewise linear function. In another embodiment, the memory 116 stores a lookup table in which the processor 104 identifies voltage threshold values stored in the lookup table using the temperature measurement as an index into the lookup table. In this embodiment, the processor 104 optionally interpolates between entries in the lookup table to identify the low battery voltage threshold if a measured temperature value does not match an exact entry value in the lookup table.

While the graph 300 in FIG. 3 depicts one example of the temperature-dependent low battery voltage threshold 304, the precise voltage threshold levels for different operating temperatures may vary in different medical device embodiments. Furthermore, a temperature-dependent low battery threshold may be formed from a single linear function, a piecewise linear function with two or more segments, or a non-linear function that adjusts the low battery voltage threshold over a temperature range.

Referring again to FIG. 1, the user I/O peripherals 140 include input devices and output devices that enable user interaction with the medical device 100. Examples of input devices include touchpads and touchscreen inputs, buttons, switches, dials, and the like. At least some types of input devices receive electrical power from the battery 128, either directly or via drive circuitry in the processor 104. The output devices include display devices such as LCD or OLED display screens, indicator lights, audio output speakers, electromechanical actuators for haptic feedback devices, and the like, and these output devices also draw electrical power from the battery 128 directly or via drive circuitry in the processor 104.

The wireless transceiver 144 is, for example, a Bluetooth, Bluetooth Low Energy (BLE), IEEE 802.11 “Wi-Fi”, Near Field Communication (NFC), cellular, or other wireless transceiver that enables the medical device 100 to perform wireless communication with external computing devices including, but not limited to, smartphones, personal computers (PCs), and network services via a data network. In one non-limiting embodiment, the wireless transceiver 144 is implemented as a BLE transceiver with an antenna that contained within the housing 50. The wireless transceiver 144 receives electrical power from the battery 128 either directly or via drive circuitry in the processor 104. In some medical device embodiments, the wireless transceiver 144 draws a substantial level of power from the battery 128 during operation, and in particular during radio transmission operations. The wireless transceiver 144 is an optional component that need not be included in every embodiment of a medical device since some medical devices are not configured for wireless communication with external computing devices.

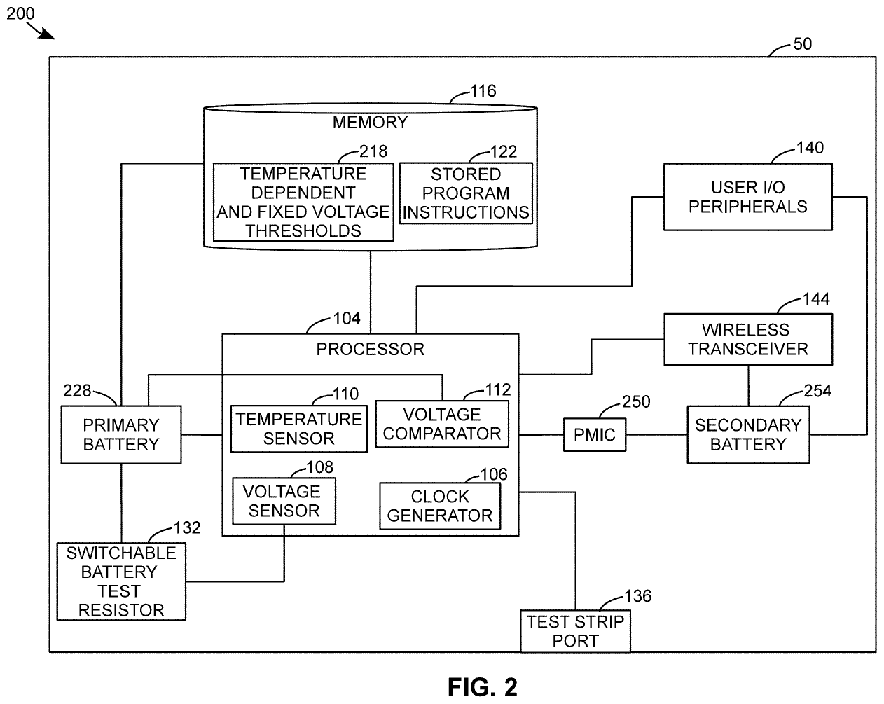

FIG. 2 depicts a schematic diagram of another battery-powered medical device 200. The medical device 200 includes some common elements to the medical device 100 including a housing 50, processor 104, memory 116, user I/O peripherals 140, and wireless transceiver 144. The medical device 200 is also depicted as a blood glucose meter that includes a test strip port 136. Unlike the medical device 100, the medical device 200 includes receptacles for two different replaceable batteries, which are depicted as a primary battery 228 and a secondary battery 254 that are both electrically connected to the medical device 200. In the configuration of FIG. 2, the primary battery 228 provides electrical power to the processor 104, including components that generate electrical test signals for the test strip port 136, and the memory 116. The secondary battery 254 provides electrical power to drive the wireless transceiver 144 and the user I/O peripherals 140. In the medical device 200, the processor 104 uses the voltage sensor 108 and switchable battery test resistor 132 to measure the voltage level of the primary battery 228 in a similar manner to that described above in FIG. 1, while a separate power management integrated circuit (PMIC) 250 provides voltage measurements of the secondary battery 254 to the processor 104. In the illustrative example of FIG. 2, both the primary battery 228 and the secondary battery 254 are lithium batteries that are available commercially as a CR2032 coin cell batteries with a nominal 3 V voltage level in a fully-charged battery.

In the embodiment of FIG. 2, the processor 104 also includes the clock generator 106, voltage sensor 108, temperature sensor 110, and the voltage comparator 112. In the medical device 200, the voltage comparator 112 is only connected to the primary battery 228. However, in an alternative configuration a second voltage comparator is connected to the secondary battery, or a multiplexer connects the voltage comparator 112 to both the primary battery 228 and the secondary battery 254 at different times.

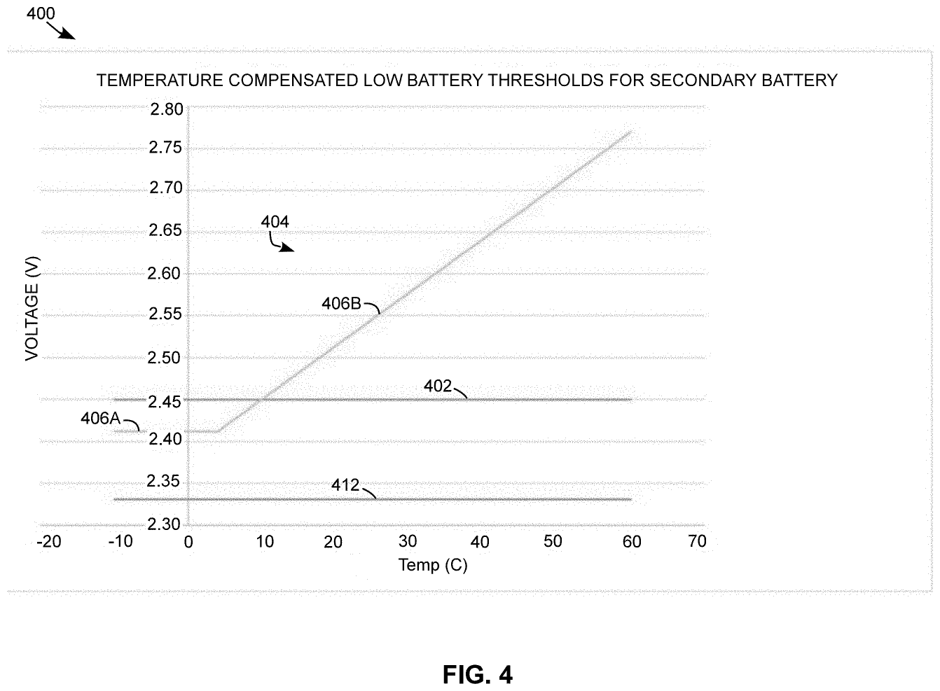

In the embodiment of FIG. 2, the primary battery 228 and the secondary battery 254 may generate different voltage levels during the course of operation of the medical device 200. The memory 116 stores battery voltage threshold data 218 that are similar to the battery thresholds 118 in the medical device 100, but optionally include separate sets of fixed and temperature-dependent low battery voltage threshold values for the primary battery 228 and the secondary battery 254, although in some embodiments both the primary battery 228 and the secondary battery 254 use the same voltage threshold values. In the illustrative embodiment of FIG. 2, the battery threshold data 218 include two different temperature-dependent low battery voltage thresholds that are used to detect low voltage conditions in the primary battery 228 and the secondary battery 254 based on a temperature measurement. In the embodiment of FIG. 2, the medical device 200 uses the temperature-dependent low battery voltage threshold 304 that is depicted above in FIG. 3 for the primary battery 228, and a second temperature-dependent low battery voltage threshold for the secondary battery 254, which is depicted in FIG. 4.

Referring to FIG. 4, a graph 400 depicts a temperature-dependent low battery voltage threshold 404 and a minimum operating voltage threshold 412 for the secondary battery 254. In the graph 400, the temperature-dependent low battery threshold 404 is a piecewise linear function that includes a first segment 406A that establishes a low battery threshold voltage of approximately 2.41V for colder operating temperatures in a range of −10 C to 5 C. A second segment 406B is another linear segment with a positive slope in relation to the operating temperature that increases the low battery voltage threshold level as the temperature increases from 5 C to 60 C in the operating temperature ranges of the medical device 100. In the illustrative example of FIG. 4, the low battery voltage threshold and minimum operating voltage thresholds for the secondary battery 254 are lower than for the primary battery 228 at a given temperature. The memory 116 stores temperature-dependent low battery voltage data 218 that also corresponds to a piecewise linear function to enable identification of the low battery voltage threshold based on the temperature measurement. During operation, if the processor 104 and PMIC 250 measure a secondary battery voltage level that exceeds the temperature-dependent low battery threshold 404, then the medical device 200 continues with normal operation, while any voltage measurement that is below the temperature-dependent low battery voltage threshold 404 at the measured temperature but also above the minimum operating voltage threshold 412 enables the medical device 200 to continue with normal operation while the processor 104 generates a low battery indicator to alert the user that the secondary battery 254 is approaching the point of replacement. The processor 104 also performs the same low battery voltage detection operation for the primary battery 228 using the temperature-dependent low battery voltage threshold 304 as described above.

The temperature-dependent low battery threshold 404 is lower than a prior-art fixed low battery threshold 402 (shown for reference) at lower temperatures of −10 C up to 10 C, and is higher than the fixed low battery threshold 402 at higher temperatures above 10 C up to 60 C. As such, the temperature-dependent low voltage threshold 404 reduces the occurrences of false-positive low battery voltage detections for the secondary battery 254 at lower temperatures and reduces the occurrences of false-negative failures to detect low battery conditions at higher temperatures. The temperature-dependent low battery threshold 404 is also greater than the minimum operating voltage threshold 412 for the secondary battery 254 at any temperature in the operating range.

In the medical device 200, the memory 116 stores parameters that describe the piecewise linear functions, such as slope, Y-intercept, and breakpoints between segments of the piecewise linear functions, and the processor 104 calculates the low battery voltage threshold using a temperature measurement from the temperature sensor 110 as the independent variable in the piecewise linear function for both the primary battery 228 and the secondary battery 254 using the selected parameters for both of the temperature-dependent low battery voltage thresholds. In another embodiment, the memory 116 stores one or more lookup tables in which the processor 104 identifies voltage threshold values stored in the lookup table using the temperature measurement as an index into the lookup table. In this embodiment, the processor 104 optionally interpolates between entries in the lookup table to identify the low battery voltage thresholds if a measured temperature value does not match an exact entry value in the lookup tables.

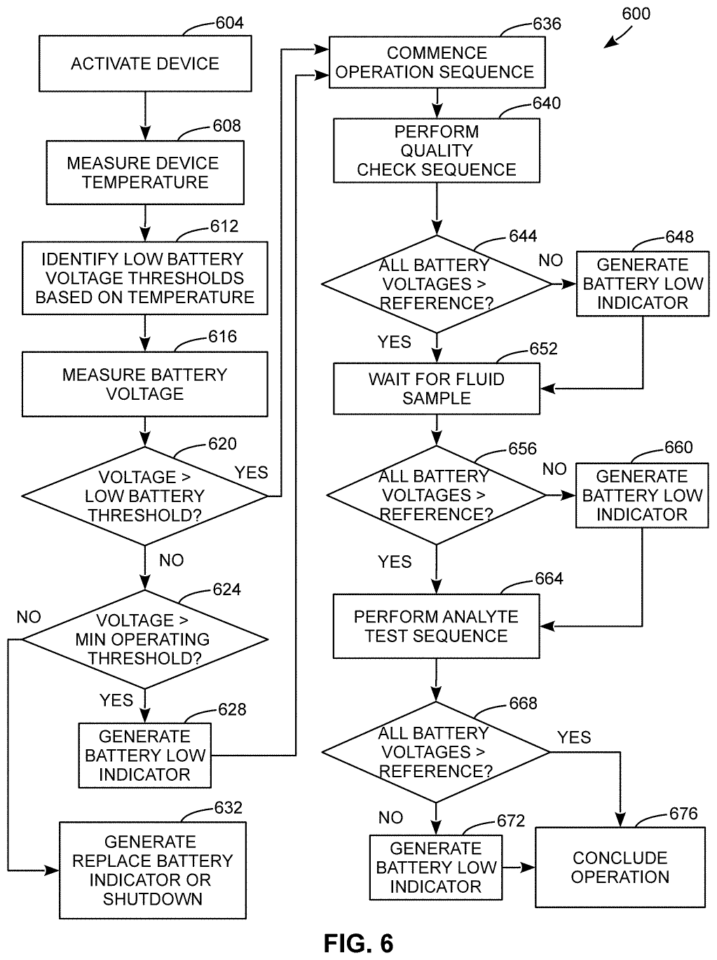

FIG. 6 depicts a block diagram of a process 600 for detecting low battery conditions in a medical device. In particular, the process 600 is applicable to the medical device 100 using a single battery 128 and to the primary battery 228 and secondary battery 254 in the medical device 200. These medical devices and batteries are referred to interchangeably in the context of the process 600 unless otherwise noted herein. In the description below, a reference to the process 600 performing a function or action refers to the operation of a processor to execute stored program instructions to perform the function or action in association with other components of a medical device.

The process 600 begins with activation of the medical device (block 604). In the medical devices 100/200, the processor 104 activates in a wakeup from hibernation mode if the medical device has been idle or in a reset mode if the main battery 128/228 has been replaced. In either mode, the processor 104 operates in a low power state at a reduced frequency clock speed controlled by the clock generator 106 to perform initial battery tests and other startup procedures prior to commencing an operation sequence to perform an analyte test or other operation.

The process continues as the processor 104 uses the temperature sensor 110 to measure a temperature within the housing 50 of the medical device 100/200 that corresponds to the temperature of the main battery 128/228 and the secondary battery 254 of the medical device 200 (block 608). As described above, the processor 104 also identifies the low battery voltage threshold for the primary battery 128/228 and, in the medical device 200, the secondary battery 254 using the temperature measurement and the temperature-dependent threshold data 118/218 (block 612). The processor 104 also measures the voltage levels of the main battery 128/228 using the voltage sensor 108 and, in the medical device 200, the voltage of the secondary battery 254 using the PMIC 250 (block 616). The temperature sensing and battery voltage measurement operations described above with reference to blocks 608 and 616 may be performed in any order or concurrently.

During the process 600, if the measured voltage level of the primary battery 128/228 or the secondary battery 254 is less than the identified temperature-dependent low battery voltage threshold (block 620), then the processor 104 further identifies if the measured voltage level also exceeds the predetermined operating voltage threshold (block 624). If the measured voltage level of the primary battery 128/228 or the secondary battery 254 is also below the corresponding minimum operating voltage threshold, then the processor 104 generates a replace battery indicator output or shuts down to the medical device 100/200 immediately (block 632). In the medical devices 100/200 the processor 104 operates a display screen, indicator light, audio output device, or other output device user I/O peripherals 140 to indicate the need to replace the battery 128 or the batteries 228 and 254, and the processor 104 prevents further operation of the medical device 100/200. If the measured voltage level is below the dead battery threshold then the processor 104 immediately shuts down the medical device 100/200.

During the process 600, if the measured voltage level of the primary battery 128/228 or the secondary battery 254 is less than the identified temperature-dependent low battery voltage threshold (block 620), but the processor 104 further identifies that the measured voltage level exceeds the predetermined minimum operating voltage threshold (block 624), then the processor 104 generates a low battery condition output and continues with a standard operation sequence of the medical device 100/200 (block 628). In the medical devices 100/200 the processor 104 operates a display screen, indicator light, audio output device, or other output device user I/O peripherals 140 to indicate that the battery 128 or one or both of the batteries 228 and 254 are in a low charge state, but the batteries do not need immediate replacement for the medical device 100/200 to perform an operation sequence.

If the measured voltage level of battery or batteries in the medical device 100/200 is greater than the temperature-dependent low battery voltage threshold (block 620) or if the medical device 100/200 generates a low battery indicator but the battery or batteries are greater than the minimum operating voltage threshold (block 628), then the process 600 continues as the medical device 100/200 commences an operation sequence (block 636). As used herein, the term “operation sequence” refers to an action or series of actions that the medical device 100/200 performs during normal operation when the battery or batteries can supply sufficient electrical power to enable performance of the operation sequence. In the medical devices 100/200 the processor 104 transitions operation to a higher power mode using a higher frequency clock signal from the clock generator 106, and the processor 104 activates other components in the medical device 100/200 that increase the load applied to the battery 128 or batteries 228 and 254 during the operation sequence. In the illustrative example of FIG. 6, the operation sequence described below is a measurement sequence for detection of an analyte in a fluid sample, such as a blood glucose measurement. In particular, the processor 104 uses the voltage comparator 112 to identify voltage droops in the primary battery 128/228 during the operation sequence when the primary battery 128/228 experiences increased load levels. However, other medical devices perform different specific operation sequences that can also produce voltage droops in a similar manner that of the medical devices 100/200, and those of skill in the art will recognize that the process 600 is also applicable these medical devices.

During the process 600, the processor 104 performs a quality check sequence in response to insertion of a test strip into the test strip port 136 (block 640). During the quality check sequence the processor 104 applies a series of electrical signals to the test strip to ensure that the test strip has not been damaged and the processor 104 further confirms that other components in the meter 100/200 are also operable. The voltage comparator 112 generates a voltage comparison between the reference voltage and the voltage level of the primary battery 128/228 during each clock cycle of the clock generator 106 during the quality check sequence. If every voltage comparison indicates that the voltage level of the primary battery 128/228 is greater than the reference voltage (block 644), then the processor 104 identifies no voltage droops during the quality check and continues to the wait for fluid sample sequence. If, however, the voltage comparator 112 generates one or more voltage comparisons in which the voltage of the primary battery 128/228 drops below the reference voltage during one or more clock cycles, then the processor 104 detects one or more voltage droops (block 644) and the processor 104 generates the battery low indicator (block 648). In the medical device 100/200 the processor 104 generates the battery low indicator in the same manner as described above with reference to the processing of block 628. Furthermore, if the medical device 100/200 has already generated the battery low indicator at any point during the process 600, then the previous battery low indicator remains active during the remainder of the operation sequence and other portions of the process 600.

The process 600 continues as the processor 104 performs a wait for fluid sample operation in which the processor 104 monitors the test strip to detect when a fluid sample, such as a blood sample, is applied to the test strip (block 652). The voltage comparator 112 continues to generate a voltage comparison during each clock cycle. If every voltage comparison indicates that the voltage level of the primary battery 128/228 is greater than the reference voltage (block 656), then the processor 104 identifies no voltage droops during wait for fluid sample operation and continues to perform an analyte test sequence. If, however, the voltage comparator 112 generates one or more voltage comparisons in which the voltage of the primary battery 128/228 drops below the reference voltage during one or more clock cycles, then the processor 104 detects one or more voltage droops (block 656) and the processor 104 generates the battery low indicator (block 660).

The process 600 continues as the processor 104 performs an analyte test sequence operation in which the processor 104 applies a sequence of electrical signals to electrodes in the after the test strip receives the fluid sample, such as a blood sample, to detect the presence of an analyte, such as glucose, and to provide the results to a user via one or both of the user I/O peripherals 140 and the wireless transceiver 144 (block 664). The voltage comparator 112 continues to generate a voltage comparison during each clock cycle. If every voltage comparison indicates that the voltage level of the primary battery 128/228 is greater than the reference voltage (block 668), then the processor 104 identifies no voltage droops during the analyte test sequence and the processor 104 concludes the analyte test sequence operation (block 676). If, however, the voltage comparator 112 generates one or more voltage comparisons in which the voltage of the primary battery 128/228 drops below the reference voltage during one or more clock cycles, then the processor 104 detects one or more voltage droops (block 668) and the processor 104 generates the battery low indicator (block 672). After concluding operation, the analyte meter 100/200 may remain activated and the user I/O devices 140 continue to provide the low battery indicator if one has been generated during one or more of the low battery checks that occur during the process 600 (block 676). The medical device 100/200 may remain activated to perform another operation sequence or to perform a different operation, such as uploading stored blood glucose data to an external computing device using the wireless transceiver 144. The processor 104 optionally measures the battery voltage of the battery 128 or batteries 228 and 254 using the temperature-dependent low battery voltage threshold prior to each subsequent operation sequence to continue to identify low battery conditions during operation of the medical device 100/200.

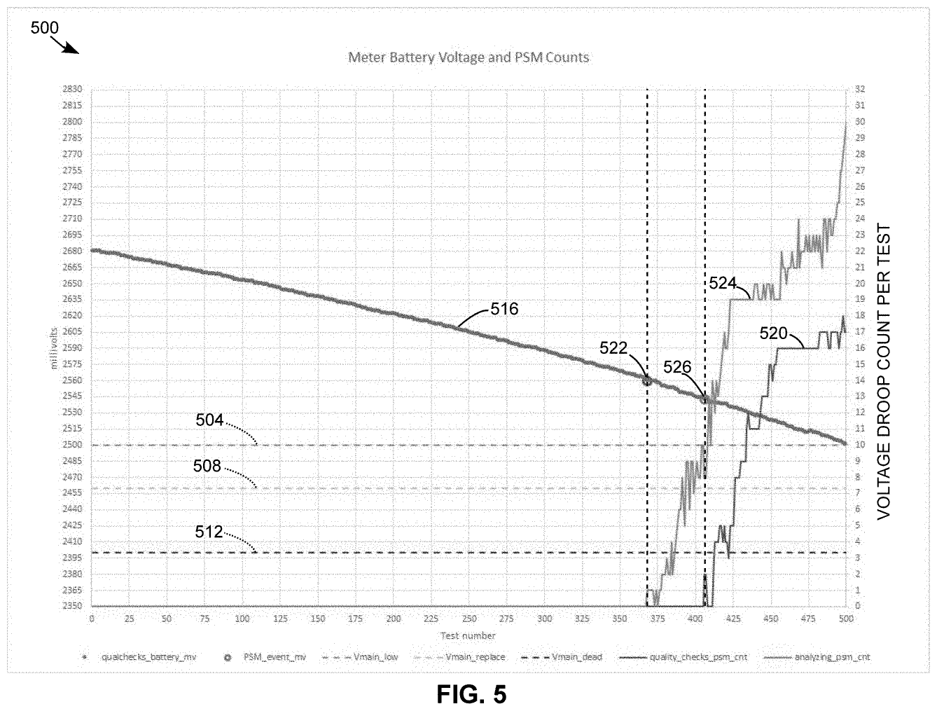

As described above, the medical device 100/200 and the process 600 implement two different techniques to identify low battery conditions, namely the use of a temperature-dependent voltage low battery threshold with direct voltage measurements of one or more batteries prior to the operation sequence and the use of the voltage comparator to identify voltage droops in a primary battery during the operation sequence. FIG. 5 depicts a graph 500 that shows a series of analyte measurement tests performed in the embodiment of the medical device 200 based on the primary battery 228 that discharges during the series of tests, although the medical device 100 using a single battery 128 produces similar results to those of the graph 500. Each test number in the graph 500 corresponds to one activation of the test meter and execution of the operation sequence to test for an analyte in a fluid sample. The graph 500 includes voltage thresholds 504, 508, 512, a nominal battery voltage measurement curve 516, and measurements of voltage droops 520 and 524 that occur during the quality check and analyte test sequences, respectively. The thresholds 504, 508, and 512 depict the low battery voltage threshold, minimum operating voltage threshold, and dead battery voltage threshold, respectively. As described above, the medical devices 100/200 identify the low battery voltage threshold based on temperature, and the low battery voltage 504 is shown for a fixed temperature used during testing for illustrative purposes. The voltage measurement curve 516 depicts a gradual decrease in the nominal voltage of the primary battery 228 that the processor 104 measures using the voltage sensor 108 while the primary battery 228 is in a lightly loaded state. The voltage droop curves 520 and 524 depict the total number of voltage droops that the processor 104 detects during either the quality check (520) or the analyte test sequence (524) of a single test sequence. While the process 600 also includes the wait for fluid drop portion of the operation sequence, voltage droops during this portion of the sequence occur less frequently and are omitted from FIG. 5 for simplicity. The graph 500 depicts that the number of detected voltage droops generally increases as the battery discharges over a number of test sequences, although the voltage droop count may vary between individual test sequences. In particular, at reference 522 the analyte test sequence curve 524 experiences the first voltage droop, while the standard battery voltage curve 516 is still well above the low battery voltage threshold 504. Similarly, at reference 526 the quality check curve 520 experiences the first voltage droop, while the while the standard battery voltage curve 516 is still exceeds the low battery voltage threshold 504. As depicted in FIG. 5, the detection of voltage droops enables the processor 104 to detect a low battery condition at an earlier time during operation compared to only measuring the nominal voltage of a battery. Similarly, the temperature-dependent low battery voltage thresholds increase the accuracy of identifying if the nominal voltage of a battery actually indicates a low battery condition during operation of the medical device.

While the embodiments described herein use both the temperature-dependent low battery voltage thresholds and the detection of voltage droops during an operation sequence to improve the accuracy of detecting low battery conditions, those of skill in the art will recognize that these features may be implemented independently from one another. For example, one alternative embodiment of a medical device may use the temperature-dependent low battery voltage thresholds described herein for detection of low battery conditions over a wide range of operating temperatures without further detecting voltage droops. Similarly, another embodiment of a medical device implements the voltage droop detection described herein while either not measuring the nominal battery voltage or using a prior art fixed voltage threshold to detect low battery conditions. However, the two techniques described herein provide particular advantages to the medical devices 100/200. As describe above, the first method using the ADC in the voltage sensor 108 returns a digital value that can then be used to compensate the battery voltage for temperature. The processor 104 incorporates a single ADC, but uses a multiplexer to select different inputs to measure, including electrodes in a test strip. When the medical device 100/200 is not performing time critical measurements, the ADC can be used to measure the battery voltage. When the processor 104 performs time critical measurements, such as measuring the voltages and currents of the analyte measurement test strip during a blood glucose or other analyte measurement, the processor 104 cannot interrupt this critical timing to measure the battery voltage. The second method that employs the voltage comparator 112 provides the yes/no status of the primary battery 128 and does not affect the timing of the processor 104. Thus, the processor 104 is configured to check for voltage droops based on a status flag received from the voltage comparator 112 after the time critical measurements have been completed and determine if the battery voltage dropped below the reference voltage while the processor was performing other time critical functions. As such, the medical devices 100/200 can monitor one or more batteries during both the device initialization and idle periods and during operation sequences to improve the detection of low battery conditions.

This disclosure is described in connection with what are considered to be the most practical and preferred embodiments. However, these embodiments are presented by way of illustration and the scope of protection is not intended to be limited to the disclosed embodiments. Accordingly, one of skill in the art will realize that this disclosure encompasses all modifications and alternative arrangements within the spirit and scope of the disclosure and as set forth in the following claims.

What is claimed is:

1. A method for operating a medical device comprising:
activating a processor in the medical device, the processor receiving electrical power from a battery electrically connected to the medical device;
measuring, with the processor, a temperature within a housing of the medical device;
identifying, with the processor, a first low battery voltage threshold based on the temperature;
measuring, with a voltage sensor operatively connected to the processor, a first voltage level of the battery;
commencing an operation sequence of the medical device after measuring the first voltage level of the battery;
generating, with a voltage comparator operatively connected to the processor, a plurality of voltage comparisons between a reference voltage level and a voltage level delivered from the battery during the operation sequence; and
generating, with the processor, an output using an output device in the medical device indicating a low battery condition in response to at least one of:
a) the first voltage level of the battery being less than the first low battery voltage threshold and above a predetermined minimum operating voltage threshold, the predetermined operating voltage threshold being less than the first low battery voltage threshold; or
b) at least one voltage comparison in the plurality of voltage comparisons indicating the voltage level of the battery is less than the reference voltage level during the operation sequence.

2. The method of claim 1, the identifying of the first low battery voltage threshold further comprising:
identifying, with the processor, the first low battery voltage using a predetermined piecewise linear function stored in a memory of the medical device.

3. The method of claim 2, wherein the memory stores parameters of the piecewise linear function and the processor calculates the first low battery voltage using the parameters.

4. The method of claim 2, wherein the memory stores a lookup table corresponding to the piecewise linear function and the processor identifies the first low battery voltage using the lookup table.

5. The method of claim 1, the operation sequence further comprising:
a quality check process;
a wait for fluid sample process; and
an analyte test sequence process, wherein the voltage comparator generates the plurality of voltage comparisons during each of the quality check process, the wait for fluid sample process, and the analyte test sequence process.

6. A medical device comprising:
a housing configured to hold:
a processor;
a memory operatively connected to the processor;
a voltage comparator operatively connected to the processor;
a temperature sensor operatively connected to the processor;
an output device operatively connected to the processor; and
a receptacle configured to be electrically connected to a battery, the receptacle being operatively connected to the processor, the memory, and the voltage comparator; and
the processor being configured to execute stored program instructions in the memory to:
activate to receive electrical power from the battery;
measure, with the temperature sensor, a temperature within the housing;
identify a first low battery voltage threshold based on the temperature;
measure, with the voltage sensor, a first voltage level of the battery;
commence an operation sequence of the medical device after the measurement of the first voltage level of the battery;
generate, with the voltage comparator, a plurality of voltage comparisons between a reference voltage level and a voltage level delivered from the battery during the operation sequence; and
generate, with the output device, an output indicating a low battery condition in response to at least one of:
a) the first voltage level of the battery being less than the first low battery voltage threshold and above a predetermined minimum operating voltage threshold, the predetermined operating voltage threshold being less than the first low battery voltage threshold; or
b) at least one voltage comparison in the plurality of voltage comparisons indicating the voltage level of the battery is less than the reference voltage level during the operation sequence.

7. The medical device of claim 6, the processor being further configured to:
identify the first low battery voltage using a predetermined piecewise linear function stored in the memory.

8. The medical device of claim 7, wherein the memory stores parameters of the piecewise linear function and the processor calculates the first low battery voltage using the parameters.

9. The medical device of claim 7, wherein the memory stores a lookup table corresponding to the piecewise linear function and the processor identifies the first low battery voltage using the lookup table.

10. The medical device of claim 6, the operation sequence further comprising:
a quality check process;
a wait for fluid sample process; and
an analyte test sequence process, wherein the voltage comparator generates the plurality of voltage comparisons during each of the quality check process, the wait for fluid sample process, and the analyte test sequence process.

11. A method for operating a medical device comprising:
activating a processor in the medical device, the processor receiving electrical power from a primary battery electrically connected to the medical device;
activating, with the processor, at least one peripheral device in the medical device, the at least one peripheral device receiving electrical power from a secondary battery electrically connected to the medical device;
measuring, with the processor, a temperature within a housing of the medical device;
identifying, with the processor, a first low battery voltage threshold based on the temperature;
identifying, with the processor, a second low battery voltage threshold based on the temperature;
measuring, with a voltage sensor operatively connected to the processor, a first voltage level of the primary battery;
measuring, with the voltage sensor operatively connected to the processor, a second voltage level of the secondary battery; and
generating, with the processor, an output using an output device in the medical device indicating a low battery condition in response to at least one of:
a) the first voltage level of the primary battery being less than the first low battery voltage threshold and above a first predetermined minimum operating voltage threshold of the primary battery, the first predetermined operating voltage threshold being less than the first low battery voltage threshold; or
b) the second voltage level of the secondary battery being less than the second low battery voltage threshold and above a second predetermined minimum operating voltage threshold of the secondary battery, the second predetermined operating voltage threshold being less than the second low battery voltage threshold.

12. The method of claim 11 further comprising:
commencing an operation sequence of the medical device after measuring the first voltage level of the primary battery and the second voltage level of the secondary battery;
generating, with a voltage comparator operatively connected to the processor, a plurality of voltage comparisons between a reference voltage level and a voltage level delivered from the primary battery during the operation sequence; and
generating, with the processor, the output using the output device in the medical device indicating the low battery condition in response to at least one voltage comparison in the plurality of voltage comparisons indicating the voltage level of the primary battery is less than the reference voltage level during the operation sequence.

13. A method for operating a medical device comprising:
activating a processor in the medical device, the processor receiving electrical power from a battery electrically connected to the medical device;
commencing an operation sequence of the medical device;
generating, with a voltage comparator operatively connected to the processor, a plurality of voltage comparisons between a reference voltage level and a voltage level delivered from the battery during the operation sequence; and
generating, with the processor, an output using an output device in the medical device indicating a low battery condition in response to at least one voltage comparison in the plurality of voltage comparisons indicating the voltage level of the battery is less than the reference voltage level during the operation sequence.

14. A medical device comprising:
a processor;
a memory operatively connected to the processor;
a voltage comparator operatively connected to the processor;
an output device operatively connected to the processor; and
a receptacle configured to be electrically connected to a battery, the receptacle being operatively connected to the processor, the memory, and the voltage comparator; and
the processor being configured to execute stored program instructions in the memory to:
activate to receive electrical power from the battery;
commence an operation sequence of the medical device;
generate, with the voltage comparator, a plurality of voltage comparisons between a reference voltage level and a voltage level delivered from the battery during the operation sequence; and
generate, with the output device, an output indicating a low battery condition in response to at least one voltage comparison in the plurality of voltage comparisons indicating the voltage level of the battery is less than the reference voltage level during the operation sequence.
